(19) United States
(12) Patent Application Publication
(10) Pub. No.: US 2010/0173788 A1
(43) Pub. Date: Jul. 8, 2010

(54) BIOMARKERS FOR BREAST CANCER

(75) Inventors: Anthony Goncalves, Marseille (FR); Jean-Paul Borg, Saint Cyr-sur-mer (FR); Eric Thomas Fung, Los Altos, CA (US); Xiao-Ying Meng, Fremont, CA (US)

Correspondence Address: FOLEY AND LARDNER LLP;SUITE 500, 3000 K STREET NW, WASHINGTON, DC, 20007, US

(73) Assignee: Vermillion, Inc.

(21) Appl. No.: 11/917,766

(22) PCT Filed: Jun. 20, 2006
(86) PCT No.: PCT/US06/23954

§ 371 Date: Dec. 28, 2009

Related U.S. Application Data
(60) Provisional application No. 60/692,241, filed on Jun. 21, 2005.

(51) Int. Cl. C40B 30/00; C07K 14/00; C07K 16/00; C07K 17/00; C07C 57/30; G01N 33/53; C40B 40/10; G06N 5/02

(52) U.S. Cl. 506/7; 530/350; 530/387.1; 530/391.1; 562/496; 435/7.1; 435/7.2; 506/18; 706/54

(57) ABSTRACT

The present invention provides protein-based biomarkers and biomarker combinations that are useful in qualifying breast cancer status in a patient. In particular, the biomarkers of this invention are useful to determine metastasis-free survival of breast cancer patients. The biomarkers can be detected by SELDI mass spectrometry in serum samples fractionated by means of anion exchange chromatography. Some of the biomarkers have been identified as: Apolipoprotein A1; Apolipoprotein A2; Haptoglobin alpha 1; Transferrin; Complement C3a; and truncated forms thereof.

### INFORMATION ON RELATED APPLICATIONS

This application claims priority to U.S. Provisional Application No. 60/692,241, filed Jun. 21, 2005, which is incorporated herein by reference in its entirety.

### FIELD OF THE INVENTION

The invention relates generally to clinical diagnostics.

### BACKGROUND

Over the last few decades, advances in adjuvant chemotherapy have substantially improved the treatment of breast cancer, notably of poor prognosis early breast cancer (EBC). Prognostic factors currently used by clinicians to aid in the decision to use adjuvant chemotherapy have been reported during the consensus conferences of National Institute Health NIH and St-Gallen. These prognostic factors include clinical (age <40) and pathological (tumor size >1.0 to 20 mm, lymph node invasion, Scarff-Bloom-Richardson (SBR) grade II-III, no hormonal receptivity) parameters. However, despite appropriate loco-regional treatment and adjuvant systemic anthracycline-based chemotherapy, 30 to 50% of patients will eventually develop metastatic relapse and die. Current clinical and pathological parameters are not able to identify, among this overall poor-prognosis population, those patients who will be actually cured by standard therapies and those who will ultimately relapse. Such a failure in prediction is thought to be due to a relative inability to fully address the molecular heterogeneity inherent to cancer process and represents a major obstacle to a more personalized management of cancer disease. The increasing development and the expected availability of alternative therapies in the adjuvant setting makes crucial the identification of parameters that might more accurately predict the clinical outcome in individual patients after standard adjuvant chemotherapy.

High-throughput RNA expression profiling of tumor was recently demonstrated as a powerful method to enlighten cancer complexity and heterogeneity as well as decipher numerous pathways and molecular networks that may simultaneously operate in cancer diseases. Notably, in EBC patients, DNA microarray studies have generated transcriptional signatures that better correlate with relapse-free or overall survival than conventional prognosis criteria. Similarly, a RT-PCR based multigene assay was recently shown to accurately predict the probability of recurrence in tamoxifen-treated node negative breast cancer.

An alternative and complementary approach is to perform proteome expression analysis. Clinical proteomic profiling studies were recently boosted by development of Surface Enhanced Laser Desorption/Ionization-time of flight (SELDI-TOF) mass spectrometry (MS) which allows relatively high-throughput protein analysis of highly complex biological samples, with limited preprocessing steps. This technology combines chromatographic fractionation of the proteome using protein biochips and time of flight MS analysis that can be applied to various clinical samples, such as serum. As blood circulates through all areas of the body, it might be modified either qualitatively or quantitatively by virtually every tissue encountered. Consequently, serum may be an appropriate surrogate tissue to investigate since it may share the fingerprint of various physiologic or pathologic processes involving cancer tissue and/or host response. Coupled to appropriate bioinformatic tools, SELDI-TOF MS was recently shown as a very promising method for probing serum to identify protein patterns and/or biomarkers related to various stages and types of solid tumors, which could serve as early diagnostic markers. SELDI-TOF MS profiling of serum samples has recently gained popularity as a new promising tool that can generate diagnostic biomarkers in a broad range of cancer diseases, including ovarian cancer, prostate cancer, and breast cancer. However, application of this technique to addressing clinical questions relating to prognosis and/or therapeutic response prediction has been limited.

A means to better predict clinical outcome is needed to optimize and individualize therapeutic decisions. The present invention provides a biomarker or combination of biomarkers capable of determining breast cancer status.

### SUMMARY

In some embodiments, the invention is directed to a method for determining breast cancer status in a subject involving measuring at least one biomarker in a biological sample from the subject, wherein the at least one biomarker is selected from the group consisting of the biomarkers of Table 1; and correlating the measurement with breast cancer status. In some embodiments, the breast cancer status is relapse of breast cancer versus breast cancer free survival.

In some embodiments, the at least one biomarker is measured by capturing the biomarker with a capture reagent on an adsorbent surface of a SELDI probe and detecting the captured biomarker by laser desorption-ionization mass spectrometry. In some embodiments, the capture reagent comprises an antibody. In other embodiments, the capture reagent comprises an IMAC or CM10 sorbent. In some embodiments, the at least one biomarker is measured by immunoassay. In some embodiments, the sample is serum. In some embodiments, the correlating is performed by a software classification algorithm.

In some embodiments, the method further comprises managing subject treatment based on the status. In other embodiment the method also comprises measuring the at least one biomarker after subject management and correlating the measurement with disease progression.

In some embodiments, the invention is directed to a method for determining the course of breast cancer involving measuring, at a first time, at least one biomarker in a biological sample from the subject, wherein the at least one biomarker is selected from the group consisting of the biomarkers of Table 1, measuring, at a second time, the at least one biomarker in a biological sample from the subject; and comparing the first measurement and the second measurement; wherein the comparative measurements determine the course of breast cancer.

In other embodiments, the invention is directed to a method comprising measuring at least one biomarker in a sample from a subject, wherein the at least one biomarker is selected from the group consisting of biomarkers of Table 1.

In some embodiments, the invention is directed to a composition comprising at least one purified biomolecule selected from the biomarkers of Table 1. In other embodiments, the invention is directed to a composition comprising a biospecific capture reagent that specifically binds a biomolecule selected from the biomarkers of Table 1. In some embodiments, the biospecific capture reagent is an antibody. In other embodiments, the biospecific capture reagent is bound to a solid support. In some embodiments, the invention is directed to a composition comprising a biospecific capture reagent bound to a biomarker of Table 1.

In some embodiments, the invention is directed to a kit containing a solid support comprising at least one capture reagent attached thereto, wherein the capture reagent binds at least one biomarker selected from the group consisting of the biomarkers of Table 1 and instructions for using the solid support to detect a biomarker of Table 1. In some embodiments, the solid support comprising a capture reagent is a SELDI probe. In other embodiments, the capture reagent is an antibody. In some embodiments, the kit additionally contains a container containing at least one of the biomarkers of Table 1. In other embodiments, the kit additionally contains a strong cation exchange chromatography sorbent.

In some embodiments, the invention is directed to a kit containing a solid support comprising at least one capture reagent attached thereto, wherein the capture reagent binds at least one biomarker selected from the group consisting of the biomarkers of Table 1 and a container containing at least one of the biomarkers. In some embodiments the capture reagent is an antibody. In some embodiments, the solid support comprising a capture reagent is a SELDI probe. In other embodiments the kit further contains a strong cation exchange chromatography sorbent.

In some embodiments, the invention is directed to a software product containing code that accesses data attributed to a sample, the data comprising measurement of at least one biomarker in the sample, the biomarker selected from the group consisting of the biomarkers of Table 1 and code that executes a classification algorithm that classifies the breast cancer status of the sample as a function of the measurement. In other embodiments, the data comprises measurement of all of the biomarkers of Table 1.

In some embodiments, the invention is directed to a method comprising detecting at least one biomarker of Table 1 by mass spectrometry or immunoassay.

In other embodiments, the invention is directed to a method involving communicating to a subject a diagnosis relating to breast cancer status determined from the correlation of at least one biomarker in a sample from the subject, wherein said at least one biomarker is selected from the group consisting of the biomarkers of Table 1. In some embodiments, the diagnosis is communicated to the subject via a computer-generated medium.

In other embodiments, the invention is directed to a method for identifying a compound that interacts with a biomarker of Table 1, wherein said method involves contacting a biomarker of Table 1 with a test compound and determining whether the test compound interacts with the biomarker.

In some embodiments, the invention is directed to a method for modulating the concentration of a biomarker of Table 1 in a cell, wherein said method comprises contacting said cell with a compound that modulates the expression of the biomarker.

In other embodiments, the invention is directed to a method of treating breast cancer in a subject, comprising administering to the subject a therapeutically effective amount of a compound that inhibits expression of an up-regulated biomarker of Table 1.

In some embodiments, the invention is directed to a method of treating breast cancer in a subject, comprising administering to the subject a therapeutically effective amount of a compound that increases expression of a down-regulated biomarker of Table 1.

### BRIEF DESCRIPTION OF THE DRAWINGS

FIGS. 1A-B show differentially expressed serum proteins according to the clinical outcome. FIG. 1A shows a protein with m/z ratio of 9192 (spectra view), upregulated in patients with metastatic disease during the follow up period (M+) compared with those without metastatic disease during the follow up period (M−), whereas m/z 8936 protein is downregulated in M+ patients compared with M− patients (gel view). FIG. 1B shows other serum proteomic markers with differential expression between M+ (shaded) and M− (unshaded) patients plotted as a function of their normalized log-transformed intensities

FIGS. 2A-D show building of a multiprotein prognostic index using serum protein pattern. Fig A shows Partial Least squares (PLS)-based projection of patients according to their new C1, C2 and C ordinates. Each dot represents a patient and shading relates to the actual outcome (M+ patients are shaded and M− patients are unshaded). FIG. 2B shows a probability graph. The probability of metastatic relapse was calculated for each patient using a logistic regression-based equation of C1, C2 and C3. Each dot is a patient and shading relates to the actual outcome (M+ patients are shaded and M− patients are unshaded). A probability threshold of 0.5 was chosen as cut-off to distinguish between predicted good and poor prognosis patients. FIGS. C and D show correlations between the molecular grouping based on the multiprotein index and the occurrence of metastatic relapse in the learning (C) and the leave-one-out cross-validated (D) set of samples.

FIGS. 3A-B show Kaplan-Meier analysis of the Metastasis-Free Survival (A) and Overall Survival (B) according to the serum multiprotein-based classification.

### DETAILED DESCRIPTION

### 1. Introduction

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

### 2. Biomarkers for Breast Cancer

### 2.1. Biomarkers

This invention provides polypeptide-based biomarkers that are differentially present in subjects having breast cancer. The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The biomarkers were discovered using SELDI technology employing ProteinChip arrays from Ciphergen Biosystems, Inc. (Fremont, Calif.) (“Ciphergen”). Serum samples were collected from a population of 81 high-risk EBC patients receiving adjuvant chemotherapy. Serum samples collected after surgery and before any specific adjuvant treatment were subfractionated by combining anion exchange chromatography and retention on chromatographic ProteinChip arrays and analyzed by time of flight-based mass spectrometry. The spectra thus obtained were analyzed by CiphergenExpress™ Data Manager Software with Biomarker Wizard. Proteins differentially expressed according to the metastatic outcome were selected and subjected to biostatistical analysis combining supervised PLS projection and logistic regression modeling. This data analysis is described in detail in the Example. A 40-protein index comprising the biomarkers of Table 1 was generated that correctly predicted the clinical outcome, long-term metastasis-free survival versus metastatic relapse, in 83% of patients and identified in this population 2 classes of patients (“good prognosis” and “poor prognosis”) with highly significant difference in 5-year metastasis-free survival and overall survival. This method is described in more detail in the Example Section.

The biomarkers thus discovered are presented in Table 1. The “ProteinChip assay” column refers to chromatographic fraction in which the biomarker is found, the type of biochip to which the biomarker binds and the wash conditions, as per the Example. The binding and washing buffer for CM10 is 100 mM NaAcetate, pH 4.0, and the binding and washing buffer for IMAC30 is 50 mM Tris, pH 8.0+500 mM NaCl.

TABLE 1
Biomarkers

| | | Up or down regulated in | |
|---|---|---|---|
| | | breast cancer patients with | |
| Marker | P-Value | subsequent metastatic relapse | ProteinChip ® assay |
| M2677 | 0.00387262 | down | F1, CM10, binding and washing |
| | | | buffers are 100 mM NaAcetate, |
| | | | pH 4.0 |
| M3093 | 0.03325257 | down | F1, CM10, binding and washing |
| | | | buffers are 100 mM NaAcetate, |
| | | | pH 4.0 |
| M4159 | 0.04610237 | down | F6, CM10, binding and washing |
| | | | buffers are 100 mM NaAcetate, |
| | | | pH 4.0 |
| M4833 | 0.04110057 | down | F4, IMAC, binding and washing |
| | | | buffers are 50 mM Tris, |
| | | | pH 8.0 + 500 mM NaCl |
| M6443 | 0.04304322 | down | F6, CM10, binding and washing |
| Apolipoprotein | | | buffers are 100 mM NaAcetate, |
| C1 (truncated) | | | pH 4.0 |
| M6641 | 0.04015743 | down | F6, CM10, binding and washing |
| | | | buffers are 100 mM NaAcetate, |
| | | | pH 4.0 |
| M6647 | 0.02807024 | down | F6, CM10, binding and washing |
| Apolipoprotein | | | buffers are 100 mM NaAcetate, |
| C1 | | | pH 4.0 |
| M6938 | 0.03487586 | down | F4, CM10, binding and washing |
| | | | buffers are 100 mM NaAcetate, |
| | | | pH 4.0 |
| M8936 C3a | 0.01296759 | down | F1, CM10, binding and washing |
| complement | | | buffers are 100 mM NaAcetate, |
| fraction | | | pH 4.0 |
| M9179 | 0.01563289 | up | F4, CM10, binding and washing |
| | | | buffers are 100 mM NaAcetate, |
| | | | pH 4.0 |
| M9192 | 0.01195419 | up | F6, IMAC, binding and washing |
| Haptoglobin | | | buffers are 50 mM Tris, |
| alpha 1 chain | | | pH 8.0 + 500 mM NaCl |
| M9714 | 0.02359958 | down | F6, CM10, binding and washing |
| | | | buffers are 100 mM NaAcetate, |
| | | | pH 4.0 |
| M10069 | 0.03832615 | up | F1, CM10, binding and washing |
| Apolipoprotein | | | buffers are 100 mM NaAcetate, |
| A1 (fragment) | | | pH 4.0 |
| M28284 | 0.0493426 | down | F6, CM10, binding and washing |
| Apolipoprotein | | | buffers are 100 mM NaAcetate, |
| A1 | | | pH 4.0 |
| M28952 | 0.04304322 | down | F6, CM10, binding and washing |
| | | | buffers are 100 mM NaAcetate, |
| | | | pH 4.0 |
| M33513 | 0.02301425 | up | F6, CM10, binding and washing |
| | | | buffers are 100 mM NaAcetate, |
| | | | pH 4.0 |
| M50897 | 0.03246546 | down | F6, IMAC, binding and washing |
| | | | buffers are 50 mM Tris, |
| | | | pH 8.0 + 500 mM NaCl |
| M58726 | 0.01405625 | down | F4, CM10, binding and washing |
| | | | buffers are 100 mM NaAcetate, |
| | | | pH 4.0 |
| M60439 | 0.0493426 | down | F6, IMAC, binding and washing |
| | | | buffers are 50 mM Tris, |
| | | | pH 8.0 + 500 mM NaCl |
| M60681 | 0.0182895 | down | F6, IMAC, binding and washing |
| | | | buffers are 50 mM Tris, |
| | | | pH 8.0 + 500 mM NaCl |
| M66631 | 0.01332189 | up | F6, CM10, binding and washing |
| | | | buffers are 100 mM NaAcetate, |
| | | | pH 4.0 |
| M67301 | 0.02607192 | up | F6, CM10, binding and washing |
| | | | buffers are 100 mM NaAcetate, |
| | | | pH 4.0 |
| M75406 | 0.01262165 | up | F6, CM10, binding and washing |
| | | | buffers are 100 mM NaAcetate, |
| | | | pH 4.0 |
| M75983 | 0.02359958 | up | F6, CM 10, binding and washing |
| | | | buffers are 100 mM NaAcetate, |
| | | | pH 4.0 |
| M81763 | 0.04824192 | up | F4, IMAC, binding and washing |
| Transferrin | | | buffers are 50 mM Tris, |
| | | | pH 8.0 + 500 mM NaCl |
| M97000 | 0.02079724 | down | F1, IMAC, binding and washing |
| | | | buffers are 50 mM Tris, |
| | | | pH 8.0 + 500 mM NaCl |
| M104776 | 0.02947463 | down | F1, IMAC, binding and washing |
| | | | buffers are 50 mM Tris, |
| | | | pH 8.0 + 500 mM NaCl |
| M113144 | 0.03656634 | down | F1, CM10, binding and washing |
| | | | buffers are 100 mM NaAcetate, |
| | | | pH 4.0 |
| M113640 | 0.03325257 | down | F1, IMAC, binding and washing |
| | | | buffers are 50 mM Tris, |
| | | | pH 8.0 + 500 mM NaCl |
| M115741 | 0.03093903 | down | F1, IMAC, binding and washing |
| | | | buffers are 50 mM Tris, |
| | | | pH 8.0 + 500 mM NaCl |
| M118659 | 0.02947463 | down | F1, IMAC, binding and washing |
| | | | buffers are 50 mM Tris, |
| | | | pH 8.0 + 500 mM NaCl |
| M132874 | 0.0440433 | up | F6, IMAC, binding and washing |
| | | | buffers are 50 mM Tris, |
| | | | pH 8.0 + 500 mM NaCl |
| M133245 | 0.04610237 | up | F6, CM10, binding and washing |
| | | | buffers are 100 mM NaAcetate, |
| | | | pH 4.0 |
| M144252 | 0.02543373 | up | F6, CM10, binding and washing |
| | | | buffers are 100 mM NaAcetate, |
| | | | pH 4.0 |
| M144819 | 0.01405625 | up | F6, CM 10, binding and washing |
| | | | buffers are 100 mM NaAcetate, |
| | | | pH 4.0 |
| M145605 | 0.01563289 | up | F6, IMAC, binding and washing |
| | | | buffers are 50 mM Tris, |
| | | | pH 8.0 + 500 mM NaCl |
| M146237 | 0.02947463 | up | F6, IMAC, binding and washing |
| | | | buffers are 50 mM Tris, |
| | | | pH 8.0 + 500 mM NaCl |
| M157649 | 0.02027297 | up | F6, IMAC, binding and washing |
| | | | buffers are 50 mM Tris, |
| | | | pH 8.0 + 500 mM NaCl |
| M182531 | 0.00148956 | down | F1, IMAC, binding and washing |
| | | | buffers are 50 mM Tris, |
| | | | pH 8.0 + 500 mM NaCl |
| M184123 | 0.01195419 | down | F1, IMAC, binding and washing |
| | | | buffers are 50 mM Tris, |
| | | | pH 8.0 + 500 mM NaCl |

The biomarkers of this invention are characterized by their mass-to-charge ratio as determined by mass spectrometry. The mass-to-charge ratio of each biomarker is provided in Table 1 after the “M.” Thus, for example, M2677 has a measured mass-to-charge ratio of 2677. The mass-to-charge ratios were determined from mass spectra generated on a Ciphergen Biosystems, Inc. PBS II mass spectrometer. This instrument has a mass accuracy of about +/−0.15 percent. Additionally, the instrument has a mass resolution of about 400 to 1000 m/dm, where m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSII, taking the maximum and minimum mass-to-charge-ratio in the cluster, and dividing by two. Accordingly, the masses provided reflect these specifications.

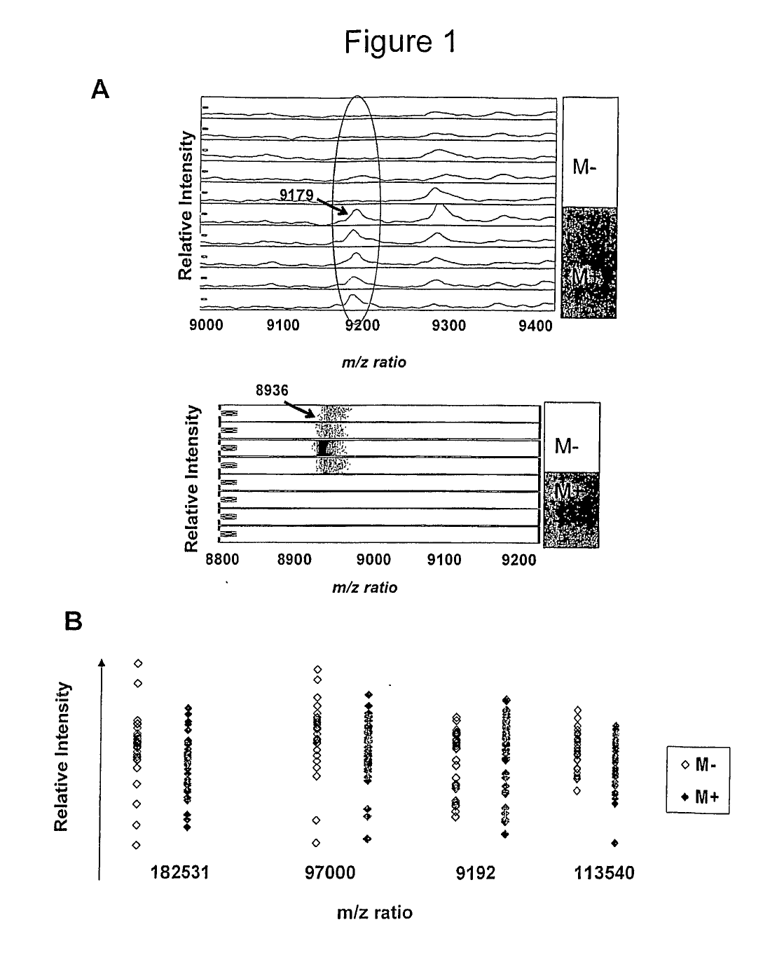

The biomarkers of this invention are further characterized by the shape of their spectral peak in time-of-flight mass spectrometry. Exemplary mass spectra showing peaks representing two of the biomarkers, M9179 and M8936, are presented in FIG. 1A.

The biomarkers of this invention are further characterized by their binding properties on chromatographic surfaces. Most of the biomarkers bind to cation exchange adsorbents (e.g., the Ciphergen® WCX or CM ProteinChip® array) after washing with 100 mM NaAc (sodium acetate) or metal chelate adsorbents (e.g., the Ciphergen® IMAC ProteinChip® array) after washing with 50 mM Tris pH 8.0+500 mM NaCl.

The identity of certain of the biomarkers of this invention has been determined and is indicated in Table 2. The amino acid sequence for each of these biomarkers is shown in the Example. The method by which this determination was made is described in the Example Section. For biomarkers whose identify has been determined, the presence of the biomarker can be determined by other methods known in the art.

TABLE 2
Identity of Biomarkers

| | | Expected molecular |
|---|---|---|
| Marker | Identity | weight |
| M6433 | Apolipoprotein C1 (truncated) | 6,432.36 |
| M6647 | Apolipoprotein C1 | 6,630.59 |
| M8936 | Complement C3a (C3a | 8,932.50 |
| | anaphylatoxin des-Arg) | |
| M9192 | Haptoglobin alpha 1 | 9,192.21 |
| M10069 | Apolipoprotein A1 (fragment) | 10,069.46 |
| M28284 | Apolipoprotein A1 | 28,078.62 |
| M81763 | Transferrin | 77,049.87 |

Because the biomarkers of this invention are characterized by mass-to-charge ratio, binding properties and spectral shape, they can be detected by mass spectrometry without knowing their specific identity. However, if desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced dissociation, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

In one embodiment, the biological source for detection of the biomarkers is serum. However, in other embodiments, the biomarkers can be detected in urine or other clinical samples including ovarian cyst fluid or ascites.

The biomarkers of this invention are biomolecules. Accordingly, this invention provides these biomolecules in isolated form. The biomarkers can be isolated from biological fluids, such as urine or serum. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

### 2.2. Use of Modified Forms of a Biomarker

It has been found that proteins frequently exist in a sample in a plurality of different forms characterized by a detectably different mass. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention (including any of Markers M2677 through M184123 of Table 1) also may be used, themselves, as biomarkers. In certain cases the modified forms may exhibit better discriminatory power in diagnosis than the specific forms set forth herein.

Modified forms of a biomarker including any of Markers M2677 through M184123 of Table 1 can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker and modified forms of it. This method also will also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified form can be accomplished by any specific detection methodology including affinity capture followed by mass spectrometry, or traditional immunoassay directed specifically the modified form. Immunoassay requires biospecific capture reagents, such as antibodies, to capture the analytes. Furthermore, if the assay must be designed to specifically distinguish protein and modified forms of protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

In another aspect this invention provides a composition comprising a biospecific capture reagent, such as an antibody, bound to a biomarker of this invention. For example, an antibody that is directed against a biomarker of this invention and that is bound to the biomarker, is useful for detecting the biomarker. In one embodiment, the biospecific capture reagent is bound to a solid support, such as a bead, a chip, a membrane or a microtiter plate.

### 3. Detection of Biomarkers for Breast Cancer

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,225,047 (Hutchens & Yip); U.S. Pat. No. 6,537,749 (Kuimelis and Wagner); U.S. Pat. No. 6,329,209 (Wagner et al.); PCT International Publication No. WO 00/56934 (Englert et al.); PCT International Publication No. WO 03/048768 (Boutell et al.) and U.S. Pat. No. 5,242,828 (Bergstrom et al.).

### 3.1. Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

### 3.1.1 SELDI

A preferred mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Urn et al., “Hydrophobic Surface Chip,” May 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyacetophenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

### 3.1.2 Other Mass Spectrometry Methods

In another mass spectrometry method, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### 3.1.3 Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### 3.1.4 General Protocol for SELDI Detection of Biomarkers for Breast Cancer

A preferred protocol for the detection of the biomarkers of this invention is as follows. The biological sample to be tested, e.g., serum, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 7, pH 5, pH 4 and pH 3. An exemplary pH7 buffer is 50 mM Hepes pH 7.0, or other suitable buffer at pH 7.0. An exemplary pH5 buffer is 100 mM NaAc buffered at pH 5.0 while an exemplary pH4 buffer is 100 mM NaAc buffered at pH 4.0. Other buffering molecules may be substituted. An exemplary pH3 buffer is 50 mM NaCitrate buffered at pH 3.0. Other buffering molecules may be substituted. (The fractions in which the biomarkers are eluted are indicated in Table 1.) Various fractions containing the biomarker are collected.

The sample to be tested (preferably pre-fractionated) is then contacted with an affinity capture probe comprising a cation exchange adsorbent (preferably a WCX or CM ProteinChip array (Ciphergen Biosystems, Inc.)) or an IMAC adsorbent (preferably an IMAC3 ProteinChip array (Ciphergen Biosystems, Inc.)), again as indicated in Table 1. The probe is washed with a buffer that will retain the biomarker while washing away unbound molecules. A suitable wash for each biomarker is the buffer identified in Table 1. The biomarkers are detected by laser desorption/ionization mass spectrometry.

Alternatively, if antibodies that recognize the biomarker are available, for example in the case of β2-microglobulin, cystatin, transferrin, transthyretin or albumin, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

### 3.2. Detection by Immunoassay

In another embodiment of the invention, the biomarkers of the invention are measured by a method other than mass spectrometry or methods that rely on a measurement of the mass of the biomarker: In another embodiment, the biomarkers of this invention are measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### 4. Determination of Subject Breast Cancer Status

### 4.1. Single Markers

The biomarkers of the invention can be used in diagnostic tests to assess breast cancer status in a subject, e.g., to diagnose breast cancer. The phrase “breast cancer status” includes any distinguishable manifestation of the disease, including the absence of disease. For example, disease status includes, without limitation, the presence or absence of disease (e.g., breast cancer v. non-breast cancer), the risk of developing disease, the stage of the disease, the progression of disease (e.g., progress of disease or remission of disease over time), the probability of either local or metastatic relapse following treatment with adjuvant therapy, metastasis free survival and the effectiveness of or response to treatment of disease. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that is actually positive. Negative predictive value is the percentage of people who test negative that is actually negative.

The biomarkers of this invention show a statistical difference in different breast cancer statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98%, at least 99% and about 100%.

Each biomarker listed in Table 1 is differentially present in breast cancer, and, therefore, each is individually useful in aiding in the determination of breast cancer status. The method involves, first, measuring the selected biomarker in a subject sample using the methods described herein, e.g., capture on a SELDI biochip followed by detection by mass spectrometry and, second, comparing the measurement with a diagnostic amount or cut-off that distinguishes a positive breast cancer status from a negative breast cancer status. The diagnostic amount represents a measured amount of a biomarker above which or below which a subject is classified as having a particular breast cancer status. For example, if the biomarker is up-regulated compared to normal during breast cancer, then a measured amount above the diagnostic cutoff provides a diagnosis of breast cancer. Alternatively, if the biomarker is down-regulated during breast cancer, then a measured amount below the diagnostic cutoff provides a diagnosis of breast cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different breast cancer statuses, as was done here, and drawing the cut-off to suit the diagnostician's desired levels of specificity and sensitivity.

### 4.2. Combinations of Markers

While individual biomarkers are useful diagnostic biomarkers, it has been found that a combination of biomarkers can provide greater predictive value of a particular breast cancer status than single biomarkers alone. Specifically, the detection of a plurality of biomarkers in a sample can increase the sensitivity and/or specificity of the test. A combination of at least two biomarkers is sometimes referred to as a “biomarker profile” or “biomarker fingerprint.” Any permutation of biomarker combinations of the biomarkers recited in Table 1 is useful for breast cancer diagnosis. For example, any combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 biomarkers is useful for determining breast cancer status.

### 4.3. Determining Risk of Developing Disease

In one embodiment, this invention provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them to a reference amount and/or pattern of biomarkers that is associated with the particular risk level.

### 4.4. Determining Stage of Disease

In one embodiment, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

### 4.5. Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers changes. Therefore, the trend of these markers, either increased or decreased over time toward diseased or non-diseased indicates the course of the disease. Accordingly, this method involves measuring one or more biomarkers in a subject at least two different time points, e.g., a first time and a second time, and comparing the change in amounts, if any. The course of disease is determined based on these comparisons.

### 4.6. Determining Metastasis Free Survival and Overall Survival

In one embodiment, this invention provides methods for determining metastasis free survival. The term “metastasis free survival” refers to a patient having lived a defined period without clinical evidence of metasasis, while “metastatic relapse” refers to clinical evidence of metastatic disease following a period of metastasis free survival. Exemplary periods of time include 12 months, 24 months, and 60 months. Long-term metastasis free survival refers to a patient who has lived without clinical evidence of metastasis for five or more years. In the Example, SELDI-TOF MS profiling of early post-operative serum from 81 high-risk EBC patients was performed to identify a protein signature correlating with metastatic relapse. By combining partial least squares and logistic regression methods, a multiprotein model comprising the biomarkers of Table 1 was built that correctly predicts outcome in 83% of patients with sensitivity, specificity, positive predictive value and negative predictive value of 87%, 75%, 84% and 80% respectively. Consistency and robustness of the model were verified using leave-one-out cross validation. Five-year metastasis free survival in “good prognosis” and “poor prognosis” patients as defined using the multiprotein index were strikingly different (83% and 22%, respectively; p <0.0001, log-rank test). In a multivariate Cox regression including conventional pathological factors and multiprotein index, only the latter retained independent prognosis significance for metastatic relapse. Major components of the multiprotein index were identified and include Haptoglobin, C3a complement fraction, Transferrin, Apolipoprotein C1 and Apolipoprotein A1.

Although conventional anthracycline-based chemotherapy improves the outcome of primary breast cancer (Polychemotherapy for early breast cancer: an overview of the randomised trials. Early Breast Cancer Trialists' Collaborative Group. Lancet, 352: 930-942, 1998), a large number of patients with high-risk EBC still relapse and die of the disease. Among this unfavorable group classically defined by lymph node invasion and/or large tumor size, young age, high SBR grade, negative hormonal receptivity (Goldhirsch, A., Wood, W. C., Gelber, R. D., Coates, A. S., Thurlimann, B., and Senn, H.-J. Meeting Highlights: Updated International Expert Consensus on the Primary Therapy of Early Breast Cancer. J Clin Oncol, 21: 3357-3365, 2003; and National Institutes of Health Consensus Development Conference Statement: Adjuvant Therapy for Breast Cancer, Nov. 1-3, 2000. J Natl Cancer Inst, 93: 979-989, 2001), a more accurate identification of patients that will be ultimately cured with conventional adjuvant chemotherapy or that will experience metastatic relapse is critical. Such an a priori knowledge could confer a higher probability of cure with currently available therapeutic strategies to certain patients, while redirecting others toward more innovative and/or aggressive strategies.

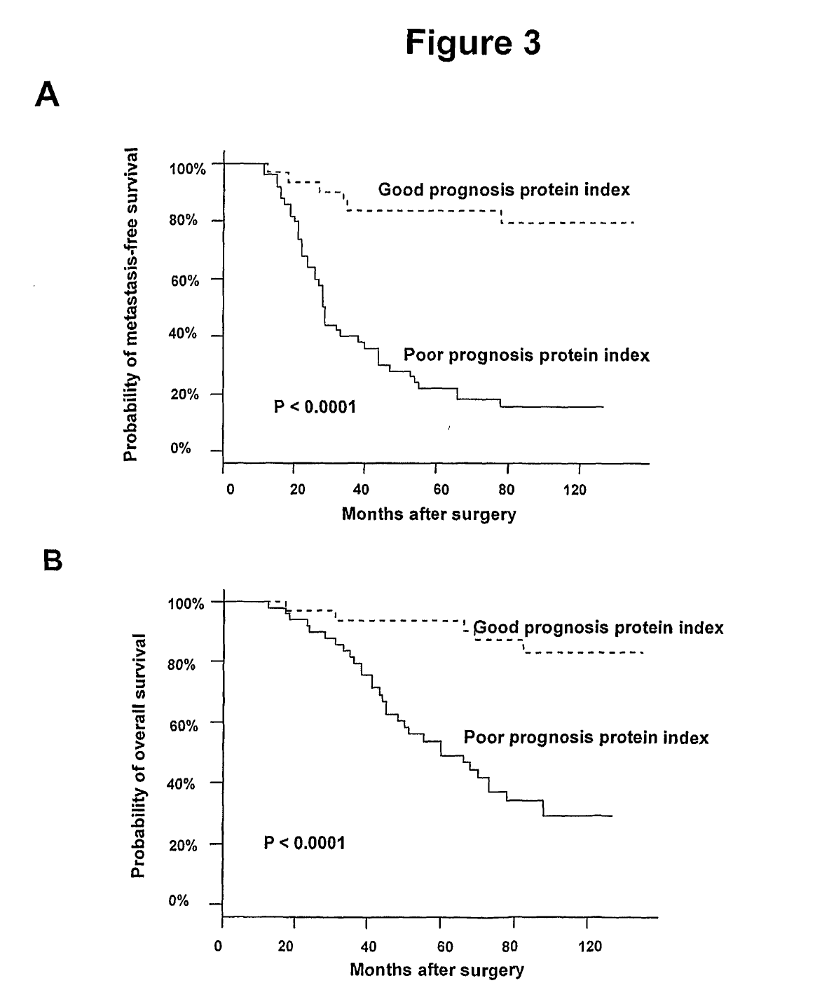

The Example describes a retrospective investigation, involving SELDI-TOF MS, of post-operative early serum proteomics from a population of 81 high-risk EBC patients receiving adjuvant chemotherapy. Serum samples collected after surgery and before any specific adjuvant treatment Were subfractionated by combining anion exchange beads and retention on chromatographic ProteinChip arrays and analyzed by time of flight-based mass spectrometry. Proteins differentially expressed according to the metastatic outcome were selected and subjected to biostatistical analysis combining supervised PLS projection and logistic regression modeling. A 40-protein index comprising the biomarkers of Table 1 was generated that correctly predicted the clinical outcome in 83% of patients and identified in this population 2 classes of patients (“good prognosis” and “poor prognosis”) with highly significant difference in 5-year metastasis-free survival and overall survival. For example, 60 months after surgery, the good prognosis protein index predicts a 88% probability of metastasis-free survival and a 94% probability of overall survival. In contrast, the poor prognosis protein index predicts a 20% probability of metastasis-free survival and a 42% probability of overall survival (see FIGS. 3A and 3B).

The multiprotein index was the only independent prognostic factor in this population when compared to conventional clinical and pathological factors that had clear prognostic significance in univariate analysis, such as lymph node invasion, pathological tumor size and grade. Some components of this multiprotein index were identified and included haptoglobin alpha 1 chain, transferrin, C3a complement fraction, apolipoprotein C1 and apolipoprotein A1.

In the Example, the patient population was retrospectively retrieved based on the availability of appropriately stored serum samples and on a sufficient follow-up (at least 6 years for disease-free surviving patients). Patient characteristics clearly displayed high-risk features with 91% of patients having lymph node invasion with a median number of 4 lymph nodes involved, 45% having grade 3 tumors, and with a median tumor size of 25 mm. Long-term metastasis-free survival and overall survival in our population were consistent with previously reported results in this subgroup of poor-prognosis early breast cancer (Bonadonna, G., Zambetti, M., and Valagussa, P. Sequential or alternating doxorubicin and CMF regimens in breast cancer with more than three positive nodes. Ten-year results. JAMA, 273: 542-547, 1995). The validity and robustness of the multiprotein index were tested using the standard leave-one-out cross-validation method.

Early post-operative serum can be informative for long term clinical outcome in breast cancer patients. Metastatic relapse after primary surgery of EBC can be thought as arising from clinically undetectable minimal residual disease after disruption of the complex interactions between cancer cells and tightly-regulated physiological tumor-control processes. These processes include stroma-generated growth factors, angiogenesis system and immune survey (Demicheli, R., Retsky, M. W., Swartzendruber, D. E., and Bonadonna, G. Proposal for a new model of breast cancer metastatic development. Ann Oncol, 8: 1075-1080, 1997; Heimann, R. and Hellman, S. Individual characterization of the metastatic capacity of human breast carcinoma. European Journal of Cancer, 36: 1631-1639, 2000; and Pupa, S. M., Menard, S., Forti, S., and Tagliabue, E. New insights into the role of extracellular matrix during tumor onset and progression. J Cell Physiol, 192: 259-267, 2002), all being potentially affected by the surgical procedure itself (Fisher, B., Gunduz, N., Coyle, J., Rudock, C., and Saffer, E. Presence of a growth-stimulating factor in serum following primary tumor removal in mice. CANCER RES., 49: 1996-2001, 1989; and Tagliabue, E., Agresti, R., Carcangiu, M. L., Ghirelli, C., Morelli, D., Campiglio, M., Martel, M., Giovanazzi, R., Greco, M., Balsari, A., and Menard, S. Role of HER2 in wound-induced breast carcinoma proliferation. The Lancet, 362: 527-533, 2003). Consequently, protein peaks participating to the multiprotein index could theoretically stem from tumor cells and/or the host response, which both have the potential to modify qualitatively or quantitatively the serum proteome. For example, the significant excess of high grade tumor and patients with ≧4 involved lymph node in the predicted “poor prognosis” class may indicate that the multiprotein index contains proteins related directly or indirectly to proliferation and invasion processes. In addition, since adjuvant chemotherapy was administered to all patients, this serum protein signature may also relate to the chemosensitivity along with the intrinsic tumor phenotype.

SELDI-TOF-based serum profiling studies reported to date have only identified as relevant biomarkers non-specific host response-generated proteins, present at rather high levels, around μg/ml (Diamandis, E. P. Analysis of serum proteomic patterns for early cancer diagnosis: drawing attention to potential problems. J Natl Cancer Inst, 96: 353-356, 2004). Although SELDI-TOF-MS has greater analytical sensitivity than this, the presence of the abundant proteins obscures the less abundant proteins. To address this problem, the Example involves prefractionating the samples using anion exchange chromatography. It is likely that other approaches to prefractionation will reveal other protein peaks. However, considering the post-operative time as a period when the tumor burden is the lowest, it is unlikely that proteins differentially expressed according to the clinical outcome may arise directly from the persistent minimal residual disease. Rather, serum multiprotein signature could correlate with post-operative host-response, including growth factor-mediated cell activation, negative immune regulation and early enhancement of the angiogenic process. As suggested by Demicheli et al (Demicheli, R., Retsky, M. W., Swartzendruber, D. E., and Bonadonna, G. Proposal for a new model of breast cancer metastatic development. Ann Oncol, 8: 1075-1080, 1997), those early post-surgical events may play a critical role in subsequent metastatic process, while determining the patients who can benefit from cytotoxic adjuvant treatment.

Some of the biomarkers of Table 1 are well-known relatively abundant host response proteins. However, some of them may yet potentially directly impact on the metastatic process. For example, haptoglobin, an acute-phase protein mainly produced in the liver has been shown to be upregulated in the serum of patients with various solid tumors (Tolson, J., Bogumil, R., Brunst, E., Beck, H., Elsner, R., Humeny, A., Kratzin, H., Deeg, M., Kuczyk, M., Mueller, G. A., Mueller, C. A., and Flad, T. Serum protein profiling by SELDI mass spectrometry: detection of multiple variants of serum amyloid alpha in renal cancer patients. Lab Invest, 84: 845-856, 2004; Ahmed, N., Barker, G., Oliva, K. T., Hoffmann, P., Riley, C., Reeve, S., Smith, A. I., Kemp, B. E., Quinn, M. A., and Rice, G. E. Proteomic-based identification of haptoglobin-1 precursor as a novel circulating biomarker of ovarian cancer. Br J Cancer, 91: 129-140, 2004; Bharti, A., Ma, P. C., Maulik, G., Singh, R., Khan, E., Skarin, A. T., and Salgia, R. Haptoglobin alpha-subunit and hepatocyte growth factor can potentially serve as serum tumor biomarkers in small cell lung cancer. Anticancer Res, 24: 1031-1038, 2004; and Kwak, J.-Y., Ma, T.-Z., Yoo, M.-J., Hee Choi, B., Kim, H.-G., Kim, S.-R., Yim, C.-Y., and Kwak, Y.-G. The comparative analysis of serum proteomes for the discovery of biomarkers for acute myeloid leukemia. Experimental Hematology, 32: 836-842, 2004), and has also been demonstrated to participate in angiogenesis, tissue remodeling and cell migration (Cid, M. C., Grant, D. S., Hoffman, G. S., Auerbach, R., Fauci, A. S., and Kleinman, H. K. Identification of haptoglobin as an angiogenic factor in sera from patients with systemic vasculitis. J Clin Invest, 91: 977-985, 1993; and De Kleijn, D. P. V., Smeets, M. B., Kemmeren, P. P. C. W., Lim, S. K., Van Middelaar, B. J., Velema, E., Schoneveld, A., Pasterkamp, G., And Borst, C. Acute-phase protein haptoglobin is a cell migration factor involved in arterial restructuring. FASEB J., 16: 1123-1125, 2002). Similarly, a proposed role for transferrin signalling has been suggested in regulating the metastatic capacity of various solid tumors including breast cancer (Cavanaugh, P. G., Jia, L., Zou, Y., and Nicolson, G. L. Transferrin receptor overexpression enhances transferrin responsiveness and the metastatic growth of a rat mammary adenocarcinoma cell line. Breast Cancer Res Treat, 56: 203-217, 1999; Cavanaugh, P. G. and Nicolson, G. L. Selection of highly metastatic rat MTLn2 mammary adenocarcinoma cell variants using in vitro growth response to transferrin. J Cell Physiol, 174: 48-57, 1998; Inoue, T., Cavanaugh, P. G., Steck, P. A., Brunner, N., and Nicolson, G. L. Differences in transferrin response and numbers of transferrin receptors in rat and human mammary carcinoma lines of different metastatic potentials. J Cell Physiol, 156: 212-217, 1993; and Nicolson, G. L., Cavanaugh, P. G., and Inoue, T. Differential stimulation of the growth of lung-metastasizing tumor cells by lung (paracrine) growth factors: identification of transferrin-like mitogens in lung tissue-conditioned medium. J Natl Cancer Inst Monogr 153-161, 1992). Transferrin was also demonstrated as promoting the angiogenic phenotype (Carlevaro, M. F., Albini, A., Ribatti, D., Gentili, C., Benelli, R., Cermelli, S., Cancedda, R., and Cancedda, F. D. Transferrin promotes endothelial cell migration and invasion: implication in cartilage neovascularization. J Cell Biol, 136: 1375-1384, 1997). Additionally, early impediment of immune surveillance, as potentially reflected in our study by a decrease in activated complement components such as C3a, may favor subsequent tumor relapse. Furthermore, it was recently shown that specific matrix metalloproteinases involved in the metastatic process were able to cleave C3b complement component thereby inhibiting subsequent complement cascade activation and protecting breast cancer cells from complement mediated injury (Rozanov, D. V., Savinov, A. Y., Golubkov, V. S., Postnova, T. I., Remacle, A., Tomlinson, S., and Strongin, A. Y. Cellular Membrane Type-1 Matrix Metalloproteinase (MT1-MMP) Cleaves C3b, an Essential Component of the Complement System. J. Biol. Chem., 279: 46551-46557, 2004).

Finally, post-translational alterations of relatively abundant host-response serum proteins without clear relevance to the metastatic process could be generated by a given tumor-specific enzymatic machinery. Rather than diluted, low level tumor-specific proteins, SELDI-analysis could detect more abundant but specifically tumor-processed host-response proteins (Zhang, Z., Bast, R. C., Jr., Yu, Y., Li, J., Sokoll, L. J., Rai, A. J., Rosenzweig, J. M., Cameron, B., Wang, Y. Y., Meng, X.-Y., Berchuck, A., van Haaften-Day, C., Hacker, N. F., de Bruijn, H. W. A., van der Zee, A. G. J., Jacobs, I. J., Fung, E. T., and Chan, D. W. Three Biomarkers Identified from Serum Proteomic Analysis for the Detection of Early Stage Ovarian Cancer. Cancer Res, 64: 5882-5890, 2004; and Paradis, V., Degos, F., Dargere, D., Pham, N., Belghiti, J., Degott, C., Janeau, J. L., Bezeaud, A., Delforge, D., Cubizolles, M., Laurendeau, I., and Bedossa, P. Identification of a new marker of hepatocellular carcinoma by serum protein profiling of patients with chronic liver diseases. Hepatology, 41: 40-47, 2004).

There has been a recent introduction of taxanes in the adjuvant treatment of EBC. Recent studies have clearly demonstrated a benefit in term of metastasis-free survival and overall survival when paclitaxel or docetaxel are associated with anthracylines in chemotherapy regimen administered after primary surgery of EBC patients with lymph node invasion (Henderson, I. C., Berry, D. A., Demetri, G. D., Cirrincione, C. T., Goldstein, L. J., Martino, S., Ingle, J. N., Cooper, M. R., Hayes, D. F., Tkaczuk, K. H., Fleming, G., Holland, J. F., Duggan, D. B., Carpenter, J. T., Frei, E., III, Schilsky, R. L., Wood, W. C., Muss, H. B., and Norton, L. Improved Outcomes From Adding Sequential Paclitaxel but Not From Escalating Doxorubicin Dose in an Adjuvant Chemotherapy Regimen for Patients With Node-Positive Primary Breast Cancer. J Clin Oncol, 21: 976-983, 2003; and Nabholtz, J. M., Pienkowski, T., Mackey, J., Pawlicki, M., Guastalla, J. P., Vogel, C., Weaver, C., Walley, B., Martin, M., Chap, L., Tomiak, E., Juhos, E., Guevin, R., Howell, A., Hainsworth, J., Fornander, T., Blitz, S., Gazel, S., Loret, C., and Riva, A. Phase III trial comparing TAC (docetaxel, doxorubicin, cyclophosphamide) with FAC (5-fluorouracil, doxorubicin, cyclophosphamide) in the adjuvant treatment of node positive breast cancer (BC) patients: interim analysis of the BCIRG 001 study. Proc Am Soc Clin Oncol, 2004). However, the taxane-anthracycline combination, although effective is probably more toxic and certainly more expensive, justifying efforts to identify patients likely to be cured by conventional antracycline-based regimen alone. In addition, the benefit of taxanes seems to be restricted to a particular subgroup with less than four lymph nodes involved, whereas no clear advantage can be shown in higher lymph node involvement. Therefore, it should remain of interest to identify among these patients those with a high probability of relapse, making them candidate to alternative strategies, potentially more effective but also more aggressive, such as dose-dense based approaches (Citron, M. L., Berry, D. A., Cirrincione, C., Hudis, C., Winer, E. P., Gradishar, W. J., Davidson, N. E., Martino, S., Livingston, R., Ingle, J. N., Perez, E. A., Carpenter, J., Hurd, D., Holland, J. F., Smith, B. L., Sartor, C. I., Leung, E. H., Abrams, J., Schilsky, R. L., Muss, H. B., and Norton, L. Randomized trial of dose-dense versus conventionally scheduled and sequential versus concurrent combination chemotherapy as postoperative adjuvant treatment of node-positive primary breast cancer: first report of Intergroup Trial C9741/Cancer and Leukemia Group B Trial 9741. J Clin Oncol, 21: 1431-1439, 2003).

### 4.7. Subject Management

In certain embodiments of the methods of qualifying or determining breast cancer status, the methods further comprise managing subject treatment based on the status. Such management includes the actions of the physician or clinician subsequent to determining breast cancer status. For example, if a physician makes a diagnosis of breast cancer, then a certain regime of treatment, such as surgery, followed by adjuvant therapy (e.g., radiotherapy, chemotherapy; antihormonal therapy, or a combination thereof) might follow. Alternatively, a diagnosis of non-breast cancer might be followed with further testing to determine a specific disease that might the patient might be suffering from. Also, if the diagnostic test gives an inconclusive result on breast cancer status, further tests may be called for.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### 4.8. Determining Therapeutic Efficacy of Pharmaceutical Drug

In another embodiment, this invention provides methods for determining the therapeutic efficacy of a pharmaceutical drug. These methods are useful in performing clinical trials of the drug, as Well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers of this invention changes toward a non-disease profile. For example, as shown in Table 1, biomarker M9197 is increased with disease, while biomarker MM2677 is decreased in disease. Therefore, one can follow the course of the amounts of these biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of the biomarkers at at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. If a treatment is effective, then the biomarkers will trend toward normal, while if treatment is ineffective, the biomarkers will trend toward disease indications.

### 5. Generation of Classification Algorithms for Qualifying Breast Cancer Status

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for breast cancer. The classification algorithms, in turn, form the base for diagnostic tests by providing diagnostic values (e.g., cut-off points) for biomarkers used singly or in combination.

### 6. Compositions of Matter

In another aspect, this invention provides compositions of matter based on the biomarkers of this invention.

In one embodiment, this invention provides biomarkers of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, this invention provides biospecific capture reagents that specifically bind a biomarker of this invention, optionally in purified form. Preferably, a biospecific capture reagent is an antibody. In one embodiment, a biospecific capture reagent is an antibody that binds a biomarker of this invention.

In another embodiment, this invention provides a complex between a biomarker of this invention and biospecific capture reagent that specifically binds the biomarker. In other embodiments, the biospecific capture reagent is bound to a solid phase. For example, this invention contemplates a device comprising bead or chip derivatized with a biospecific capture reagent that binds to a biomarker of this invention and, also, the device in which a biomarker of this invention is bound to the biospecific capture reagent.

In another embodiment, this invention provides a device comprising a solid substrate to which is attached an adsorbent, e.g., a chromatographic adsorbent, to which is further bound a biomarker of this invention.

### 7. Kits for Detection of Biomarkers for Breast Cancer

In another aspect, the present invention provides kits for qualifying breast cancer status, which kits are used to detect biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### 8. Use of Biomarkers for Breast Cancer in Screening Assays and Methods of Treating Breast Cancer

The methods of the present invention have other applications as well. For example, the biomarkers can be used to screen for compounds that modulate the expression of the biomarkers in vitro or in vivo, which compounds in turn may be useful in treating or preventing breast cancer in patients. In another example, the biomarkers can be used to monitor the response to treatments for breast cancer. In yet another example, the biomarkers can be used in heredity studies to determine if the subject is at risk for developing breast cancer.

Thus, for example, the kits of this invention could include a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers of this invention on the chip and to use these measurements to diagnose breast cancer.

Compounds suitable for therapeutic testing may be screened initially by identifying compounds which interact with one or more biomarkers listed in Table I. By way of example, screening might include recombinantly expressing a biomarker listed in Table I, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table I, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table I may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test Compounds. The ability of a test compound to interfere with a non-enzymatic (e.g., structural) function or activity of one of the biomarkers of Table I may also be measured. For example, the self-assembly of a multi-protein complex which includes one of the biomarkers of Table I may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table I may be administered to patients who are suffering from or are at risk of developing breast cancer or other cancer. For example, the administration of a test compound which increases the activity of a particular biomarker may decrease the risk of breast cancer in a patient if the activity of the particular biomarker in vivo prevents the accumulation of proteins for breast cancer. Conversely, the administration of a test compound which decreases the activity of a particular biomarker may decrease the risk of breast cancer in a patient if the increased activity of the biomarker is responsible, at least in part, for the onset of breast cancer.

In an additional aspect, the invention provides a method for identifying compounds useful for the treatment of disorders such as breast cancer which are associated with increased levels of modified forms of one or more of the biomarkers of Table 1. For example, in one embodiment, cell extracts or expression libraries may be screened for compounds which catalyze the cleavage of the full-length biomarkers of Table 1 to form truncated forms of one or more of the biomarkers of Table 1. In one embodiment of such a screening assay, cleavage of one or more of the biomarkers of Table 1 may be detected by attaching a fluorophore to one or more of the biomarkers of Table 1, which remains quenched when one or more of the biomarkers of Table 1 are uncleaved but which fluoresces when one or more of the biomarkers of Table 1 are cleaved. Alternatively, a version of one or more of the full-length the biomarkers of Table 1 may be modified so as to render the amide bond between certain amino acids uncleavable may be used to selectively bind or “trap” the cellular protease which cleaves one or more of the full-length biomarkers of Table 1 at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the progression or likelihood of a disease, e.g., breast cancer, which is associated with the increased levels of truncated forms of one or more of the biomarkers of Table 1. For example, after one or more proteins have been identified which cleave one or more of the full-length biomarkers of Table 1, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of one or more of the biomarkers of Table 1.

Compounds which impart truncated forms of the biomarkers of Table 1 with the functionality of the full-length biomarkers of Table 1 are likely to be useful in treating conditions, such as breast cancer, which are associated with the truncated form of one or more of the biomarkers of Table 1. Therefore, in a further embodiment, the invention provides methods for identifying compounds which increase the affinity of truncated forms of the biomarkers of Table 1 for their target proteases. For example, compounds may be screened for their ability to impart truncated forms of one or more of the biomarkers of Table 1 with the protease inhibitory activity of one or more of the full-length biomarkers of Table 1. Test compounds capable of modulating the inhibitory activity of one or more of the biomarkers of Table 1 or the activity of molecules which interact with one or more of the biomarkers of Table 1 may then be tested in vivo for their ability to slow or stop the progression of breast cancer in a subject.

At the clinical level, screening a test compound includes obtaining samples from test subjects before and after the subjects have been exposed to a test compound. The levels in the samples of one or more of the biomarkers listed in Table I may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table I may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table I may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with breast cancer, test compounds will be screened for their ability to slow or stop the progression of the disease.

### 9. Examples

### 9.1. Discovery of Biomarkers for Predicting Metastatic Relapse in Breast Cancer Patients Receiving Adjuvant Chemotherapy

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In this study post-operative serum from 81 breast cancer patients was studied using SELDI-TOF MS to identify a protein signature correlating with metastatic relapse.

### 9.1.1. Materials and Methods

### 9.1.1.1. Samples

The study involved a retrospective series of 81 post-operative serum samples from high-risk EBC patients, collected between 1994 and 2000 at the Institut Paoli-Calmettes (Marseille, France) with available clinical, pathological and follow-up annotations. All samples were collected with institutional approval. Selection of samples was based on the following criteria: i)—EBC with delivery of adjuvant anthracycline-based chemotherapy because of a high risk of metastatic relapse defined according to the following parameters: pathological lymph node involvement or no pathological lymph node involvement but negative hormonal receptor status or pathological tumor size >20 mm or age <40 or Grade III; ii)—availability of post-operative and pre-chemotherapy serum; iii)—no relapse after a minimal follow-up of 6 years after diagnosis or metastatic relapse within 6 years. Patients with second primary cancer or local/contralateral relapse before metastatic recurrence were excluded from the sample population. Serum was obtained within 21 days after surgery and before initiation of any other anticancer treatment. All samples were processed promptly after collection and rapidly frozen at −80° C.

### 9.1.1.2. Protein Expression Profiling

Samples were subjected to SELDI-TOF MS profiling using the ProteinChip Biomarker System as recommended by Ciphergen Biosystems (Fremont, USA). Briefly, serum samples (20 μl) were first denatured and fractionated using anion exchange chromatographic beads and pH gradient elution (pH 9/flow through, 7, 5, 4, 3, organic solvent, referred as to F1, F2, F3, F4, F5 and F6). Serum was incubated with the beads at pH 9.0 in a buffer containing 50 mM Tris pH 9.0 and 8 M urea. Proteins that did not bind the beads were eluted (the flow-through fraction), and the beads were washed with 100 ul of Tris pH 9.0 buffer. The unbound material was eluted and pooled with the flow through; this represents fraction 1. 100 ul of 100 mM Hepes pH 7.0 buffer was added to the beads, allowed to incubate, and proteins eluted. This procedure was repeated and the two pH 7.0 elutions were pooled to generate fraction 2 (F2). A similar procedure was performed to generate F3-F6. For F3, the buffer was 100 mM NaAc pH 5.0; for F4, the buffer was 100 mM NaAc pH 4.0; for F5, the buffer was 50 mM NaCitrate pH 3.0; and for F6, the buffer was 0.3% isopropanol/16.7% acetonitrile/0.1% trifluoracetic acid. Addition of a small amount of detergent (e.g. 0.1% OGP) may be included in buffers used to generate F2-F5. Aliquots of fractions (10 μl) were diluted in the appropriate chip binding buffer and bound with a randomized chip/spot allocation scheme to IMAC-Cu (buffers are 50 mM Tris pH 8.0+500 mM NaCl) and CM10 (buffers are 100 mM NaAc pH 4.0) ProteinChip arrays (See Table 1). The energy absorbing molecule (crystallization matrix) sinapinic acid was dissolved in 50% acetonitrile/0.5% trifluoroacetic acid and was promptly applied. Spotted arrays were read using the PBS IIC ProteinChip reader. All samples to be compared in a given experimental condition were processed together. For each experimental condition, arrays were read at two setting either optimized for low molecular weight (2,000-30,000) or high molecular weight (20,000-200,000) ranges.

Fractionation steps, ProteinChip array binding and matrix applications were performed using a Biomek 2000 Robot (Beckman Coulter, Fullerton Calif., USA) equipped with the ProteinChip Biomarker Integration Package. Only F1, F4 and F6 fractions that had been shown in preliminary experiments as the fractions containing the largest number of peaks were subjected to analysis. A pool of randomly spotted human serum specimens was used for monitoring the intra-assay reproducibility. External mass calibration was performed daily and instrument performances were monitored weekly, using appropriate purified protein mix.

Spectra were externally calibrated, baseline subtracted, and normalized to total ion current within m/z (mass/charge) range of 1.5-150 kDa. Qualified mass peaks (signal/noise>5; cluster mass window at 0.3%) within the m/z range of 2-20 kDa (LMW) and 20-200 kDa (HMW) were selected automatically using integrated Biomarker Wizard software. The resulting Excel files containing absolute intensity and m/z ratio of protein peaks resolved were obtained and subjected to data analysis.

### 9.1.1.3. Analysis of Proteomic Data

Logarithmic transformation was applied to the peak intensity before analysis for biomarker discovery. Protein peaks resolved from each experimental condition were tested for differential expression between patients with metastatic relapse (M+) and long-term metastasis-free survivors (M−) using Mann-Whitney test. Differentially expressed proteins were defined as those with a p-value <0.05.

Selected proteins were then subjected to Partial Least Squares (PLS) projection (Nguyen, D. V. and Rocke, D. M. Tumor classification by partial least squares using microarray gene expression data. Bioinformatics, 18: 39-50, 2002; Nguyen, D. V. and Rocke, D. M. Multi-class cancer classification via partial least squares with gene expression profiles. Bioinformatics, 18: 1216-1226, 2002; and Antoniadis, A., Lambert-Lacroix, S., and Leblanc, F. Effective dimension reduction methods for tumor classification using gene expression data. Bioinformatics, 19: 563-570, 2003.). PLS is a method that reduces high dimensional multivariate data (x1, x2, . . . xn) by creating several linear combinations (herein C1, C2, and C3) that substitute for the original variables: [C1 (x1, x2, . . . , xn)=α1,0+α1,1 x1+α1,2 x2+. . . α1,n xn]; [C2 (x1, x2, . . . , xn)=α2,0+α2,1 x1+α2,2x2+. . . α2,n xn]; [C3 (x1, x2, . . . , xn)=α3,0+α3,1 x1+α3,2 x2+. . . α3,n xn]. Coefficients α were chosen in a supervised fashion to maximize covariance of C1, C2 and C3 with the phenotype to discriminate (M+ or M−). Logistic regression was applied to the new variables to build a model that calculates the probability (between 0 and 1) of displaying metastatic relapse, knowing C1, C2 and C3. A probability threshold of 0.5 was chosen as cut-off to distinguish between predicted good and poor prognosis patients. Biostatistics was performed using R software version 2.0.0.

The model was tested for consistency, robustness and validity by using the leave-one-out cross-validation class prediction method. Briefly, one withholds a sample, builds a predictor based only on the remaining samples, and predicts the class of the withheld sample. The process is repeated for each sample and the cumulative error rate is calculated.

### 9.1.1.4. Statistical Analysis

Distributions of molecular, pathological and clinical factors were compared using either the Chî2 or Fisher exact tests for categorical variables and the Wilcoxon and Mann-Whitney tests for continuous variables. Metastasis-free survival was calculated from the date of diagnosis to the time of metastasis as first event or time of last follow-up for censored patients. Overall survival was calculated from the date of diagnosis to the time of death as first event or time of last follow-up for censored patients. No patient died from cause other than breast cancer. Survival estimates were derived from Kaplan-Meier method (Kaplan, E. L. and Meier, P. Nonparametric estimation from incomplete observations. J Am Stat assoc, 53: 457-481, 1958) and compared by log-rank test. The influence of molecular grouping, adjusted for other factors, was assessed in multivariate analysis by a Cox proportional hazard model (Cox, D. R. Regression models and life table. J R Stat Soc [B] 187-220, 1972). Survival rates, odds ratio (OR) and relative risks (RR) are presented with their 95% confidence intervals (95% CI). Statistical tests were two-sided at the 5% level of significance. All statistical tests were performed using R software version 2.0.0.

### 9.1.1.5. Identification of Protein Markers by Immunodepletion

Immunodepletion experiments using commercially available antibodies against selected proteins were performed. Antibodies included mouse anti-human apolipoprotein A1 (Calbiochem 178474); sheep anti-human haptoglobin (Cortex, CR2114SP); rabbit anti-human apolipoprotein C1 (Academy Bio-Medical Company, 31A-R1a); rabbit anti-human complement C3a (Cortex CR6032RP); control mouse IgG (Sigma, 15381);); rabbit anti-human transferrin (Dako 0061), control sheep IgG (Sigma, 15131); control rabbit IgG (Sigma, 15006).

Briefly, antibody was coupled to 100 ul Aminolink Plus coupling gel (Pierce). 20 ul serum was fractionated using the EDM serum fractionation kit (Ciphergen, K100-0007). Relevant fractions, or crude serum, were diluted in binding buffer (phosphate buffered saline (PBS) containing 0.1% triton) and incubated with antibody-coupled beads at 4° C. o. The beads were then washed with PBS or PBS with triton three times, and then briefly with water. Bound material was eluted with 10 ul elution buffer (33.3% acetonitrile, 16.7% isopropanol, 0.1% trifluoroacetic acid). The elutions were pooled and applied to NP20 ProteinChip arrays (Ciphergen) with sinapinic acid as matrix.

### 9.1.2. Results

### 9.1.2.1. Patient Characteristics

Serum from 81 high-risk EBC patients receiving adjuvant chemotherapy was subjected to protein profiling using SELDI-TOF MS technology. Clinical and pathological characteristics of patient and samples are shown in Table 3. All patients had been treated by primary surgical resection and serum samples were collected post-operatively before starting any adjuvant treatment. All patients had received adjuvant chemotherapy, mostly anthracycline-based (97%), and subsequent locoregional radiotherapy. Hormonal therapy by antiestrogen (21 patients) or antiaromatase (1 patients) was administered after chemotherapy and radiotherapy when appropriate. No patients received taxane-based adjuvant treatment.

TABLE 3
Patient demographics (n = 81)

| Age | | |
|---|---|---|
| Median (range) | 49 | (27-78) |
| Less than 40 | 14 | (18%) |
| Lymph node (LN) invasion | | |
| Patients with positive LN (%) | 74 | (91%) |
| Median No of involved LN(range) | 4 | (0-26) |
| SBR grade | | |
| Grade I | 8 | (10%) |
| Grade II | 34 | (42%) |
| Grade III | 37 | (46%) |
| NA | 2 | (2%) |
| Pathological tumor size (mm) | | |
| Median (range) | 25 | (10-210) |
| Hormonal receptivity | | |
| Positive | 58 | (71%) |
| Negative | 20 | (26%) |
| NA | 3 | (4%) |
| Pathological type | | |
| Ductal | 63 | (78%) |
| Lobular | 9 | (11%) |
| Other | 9 | (11%) |

After a median follow-up of 86 months (range 20 to 115), 48 patients displayed metastatic relapse (M+) and 33 patients were long-term metastasis-free survivors (M−). Five-year metastasis-free survival and overall survival were 45.7% [95% CI 36-57.9] and 66.8% [95% CI 57.1-78.1], respectively.

### 9.1.2.2. Protein Profiling of Serum Samples

Serum samples were first fractionated using anion exchange beads. Because preliminary experiments identified fractions one, four and six as the fractions generating the largest number of resolved peaks, only those fractions were bound to CM10 and IMAC-Cu ProteinChip arrays. These six conditions (F1 CM10, F1 IMAC, F4 CM10, F4 IMAC, F6 CM10 and F6 IMAC) generated 667 protein peaks in total, ranging from 96 to 129 peaks per condition. Absolute linear and normalized log-transformed intensity values of all serum protein resolved across the sample population were determined (data not shown).

The intra-assay variation of each SELDI ProteinChip assay was determined by SELDI profiling of a mix of pooled serums from the study population, spotted randomly onto 12 of the 96 wells of the ProteinChip arrays along with the 81 analytical samples. The pooled coefficient of variance (pCV) for peak intensity was calculated for each experimental condition and had a mean of 22% (12 to 35%), in agreement with previous reports (Petricoin, E. F., Ardekani, A. M., Hitt, B. A., Levine, P. J., Fusaro, V. A., Steinberg, S. M., Mills, G. B., Simone, C., Fishman, D. A., Kohn, E. C., and Liotta, L. A. Use of proteomic patterns in serum to identify ovarian cancer. Lancet, 359: 572-577, 2002; Koopmann, J., Zhang, Z., White, N., Rosenzweig, J., Fedarko, N., Jagannath, S., Canto, M. I., Yeo, C. J., Chan, D. W., and Goggins, M. Serum diagnosis of pancreatic adenocarcinoma using surface-enhanced laser desorption and ionization mass spectrometry. Clin Cancer Res, 10: 860-868, 2004; Zhang, Z., Bast, R. C., Jr., Yu, Y., Li, J., Sokoll, L. J., Rai, A. J., Rosenzweig, J. M., Cameron, B., Wang, Y. Y., Meng, X.-Y., Berchuck, A., van Haaften-Day, C., Hacker, N. F., de Bruijn, H. W. A., van der Zee, A. G. J., Jacobs, I. J., Fung, E. T., and Chan, D. W. Three Biomarkers Identified from Serum Proteomic Analysis for the Detection of Early Stage Ovarian Cancer. Cancer Res, 64: 5882-5890, 2004.; and Petricoin, E. F., III, Ornstein, D. K., Paweletz, C. P., Ardekani, A., Hackett, P. S., Hitt, B. A., Velassco, A., Trucco, C., Wiegand, L., Wood, K., Simone, C. B., Levine, P. J., Linehan, W. M., Emmert-Buck, M. R., Steinberg, S. M., Kohn, E. C., and Liotta, L. A. Serum Proteomic Patterns for Detection of Prostate Cancer. J Natl Cancer Inst, 94: 1576-1578, 2002).

### 9.1.2.3. Identification of a Prognostic Multiprotein Signature

Among the six experimental conditions, 40 protein peaks presented a statistically significant differential expression (p<0.05 using Mann-Whitney test) between patients with metastatic outcome (M+) and metastasis-free surviving patients (M−). For example, FIG. 1A illustrates a protein of 9192 m/z ratio that was upregulated in post-operative serum of M+ patients as compared with that of M− patients, while a 8936 m/z ratio protein was downregulated. FIG. 1B provides scatter plot representation of normalized log-transformed expression of other differentially expressed proteins between M+ and M− patients.

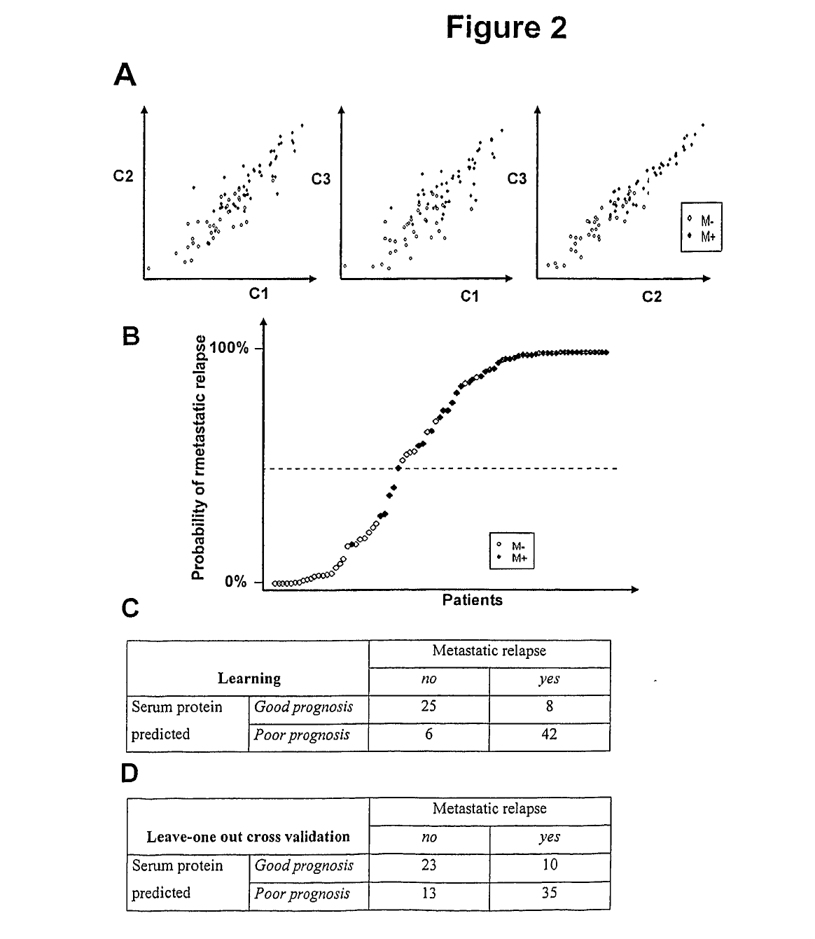

To identify a multiprotein signature that can predict metastatic relapse, a two-step biostatistic process was used. First, the dimension of the data set was reduced using the partial least squares (PLS) method. PLS allowed for generation of 3 linear combinations of the 40 protein peak intensities, creating 3 new variables C1, C2 and C3 that had been chosen in a supervised fashion to maximize the covariance of the data set with the phenotype to discriminate, i.e. metastatic outcome. FIG. 2A illustrates the projection of each sample according to its new coordinates in the C1-C2, C1-C3 and C2-C3 planes. As shown, patients with metastatic relapse were then easily separated from long-term metastasis-free survivors. Then, using a logistic regression model, an equation was built that gave for each sample the probability of metastatic relapse, knowing C1, C2 and C3. FIG. 2B shows the probability of metastatic relapse according to the multiprotein index, along with the actual outcome of patients. Samples ordered using this probability were sorted in two classes: samples with a calculated probability greater than 0.5 were assigned to the “poor prognosis” class, while those with a calculated probability less than 0.5 were assigned to the “good prognosis” class. This classifier predicted rather successfully clinical outcome: 42/50 patients (84%) in the “poor-prognosis” class displayed metastatic relapse whereas 6/31 (19%) in the “good-prognosis” class did (OR=21.87 [95% CI 6.79-70.38], p<0.0001, Fisher exact test). Thus, the multiprotein index was able to correctly predict outcome in 83% of patients with sensitivity, specificity, positive predictive value (PPV) and negative predictive value (NPV) of 87%, 76%, 84% and 81%, respectively (FIG. 2C). Consistency and robustness of the model were verified using leave-one-out cross validation. The observed correct prediction rate after cross-validation was 72% and sensitivity, specificity, NPV and PPV were 73%, 70%, 78% and 63%, respectively.

### 9.1.2.4. Multiprotein-Based Classification of Breast Cancer Samples

An analysis was conducted to search for correlations between the multiprotein-based classification and histo-clinical features of tumors. As mentioned above, there was a strong correlation with clinical outcome. As shown in FIG. 3A, the 5-year metastasis-free survival were very significantly different between the two classes of patients defined by the multiprotein index. Five-year metastasis-free survival in the “good-prognosis” class was 84% compared to 22% in the “poor-prognosis” class (p<0.0001, log-rank test). Five-year overall survival was also very largely different between these two classes (94% vs 49%; p<0.0001, log-rank test) (FIG. 3B). As shown in Table 4, tumor size, hormonal receptivity and age were not significantly different between the two prognostic classes. However, in the “poor-prognosis” class, there were significantly more patients with ≧4 involved lymph node and more patients with grade III tumors. Of note, the multiprotein index retained prognostic significance regardless of lymph node invasion. For example, the multiprotein signature classified the 37 patients with 0 to less than 4 involved axillary lymph nodes in two classes that correlated with metastasis-free survival. In the “good-prognosis” class, 1/19 patients experienced metastatic relapse as compared with 14/18 in the “poor-prognosis” class (OR=63 [95% CI 6.31-628.34], p<0.0001, Fisher exact test). The same was true for the 43 patients with 4 or more involved lymph nodes: the OR for metastasis was 8.4 (95% CI 1.72-40.9, p=0.0095, Fisher exact test) among the 32 women assigned to the “poor-prognosis” class as compared to the 11 women assigned to the “good-prognosis” class. The rate of metastasis in patients with 0 or less than 4 involved lymph nodes assigned to the “poor-prognosis” class of patients (14/18) was higher than in patients with 4 or more involved axillary lymph node assigned to the “good-prognosis” class of patients (5/11) (OR =4.2, [95% CI 0.81-21.32]). However, this difference did not reach statistical significance (p=0.11, Fisher exact test).

TABLE 4
Clinical and pathological parameters within
newly generated prognosis groups

| | “good prognosis” | “poor prognosis” | p value |
|---|---|---|---|
| | (n = 31) | (n = 50) | (Chi2 test) |
| Lymph node invasion | | | |
| 0-3 | 19 | 18 | |
| ≧4 | 11 | 32 | 0.03 |
| Pathological tumor | | | |
| size | | | |
| pT1 | 12 | 14 | |
| pT2 | 15 | 25 | 0.36* |
| pT3 | 2 | 10 | 0.2** |
| SBR grade | | | |
| I/II | 22 | 20 | |
| III | 9 | 28 | 0.02 |
| Hormonal Receptors | | | |
| Positive | 25 | 33 | |
| Negative | 4 | 16 | 0.1 |
| Age | | | |
| ≦40 | 3 | 11 | |
| >40 | 28 | 39 | 0.26 |

*pT1 vs pT2/T3
**pT1/T2 vs pT3

### 9.1.2.5. Uni- and Multivariate Analysis

An analysis was conducted to estimate the prognostic value of conventional clinical and pathological factors in our population. As expected, pathological tumor size, grade and lymph node invasion correlated with metastasis-free survival in univariate analysis (data not shown). However, in a multivariate Cox regression model that included the multiprotein index, grade (I/II vs III), pathological tumor size (pT1/pT2 vs PT3), and lymph node invasion (less than 4 vs 4 or more), (table 5) only the multiprotein index retained statistically significant association with metastasis-free survival (HR=5.6 , [95% CI 2.3-13.8], p=0.00013).

TABLE 5
Cox proportional-hazards multivariate
analyses in Metastasis-Free Survival

| Variable | Hazard ration [95% CI] | | p Value (log-rank test) |
|---|---|---|---|
| Multiprotein index | | | |
| “Good prognosis” | 1 | | |
| “Poor prognosis” | 5.6 | [2.3-13.8] | 0.00013 |
| Lymph Node invasion | | | |
| <4 | 1 | | |
| ≧4 | 1.56 | [0.8-3] | 0.19 |
| Grade | | | |
| I/II | 1 | | |
| III | 1.6 | [0.8-3.02] | 0.13 |
| Pathological tumor | | | |
| size | | | |
| pT1/T2 | 1 | | |
| pT3 | 2 | [0.9-4.2] | 0.056 |

### 9.1.2.6. Serum Protein Identities

Identities for several potential protein biomarkers participating in the multiprotein index were determined according to their m/z ratio, the fraction from which they were derived, the ProteinChip surface of capture, as well as data from previously performed serum profiling studies (E. T. Fung, unpublished data) (see Table 2). These identities were further confirmed by serum immunodepletion experiments using specific antibodies. The amino acid sequences of the biomarkers shown in Table 2 are as follows:

M6433: Apolipoprotein C-I (truncated)
(Apolipoprotein with Thr and Pro deleted from the
N-terminus)
DVSSALDKLKEFGNTLEDKARELISRIKQSELSAKMREWFSETFQKVKEK
LKIDS
M6647: Apolipoprotein C-I
TPDVSSALDKLKEFGNTLEDKARELISRIKQSELSAKMREWFSETFQKVK
EKLKIDS
M8936: Complement C3a (C3a anaphylatoxin des-Arg)
SVQLTEKRMD KVGKYPKELR KCCEDGMREN PMRFSCQRRT
RFISLGEACK KVFLDCCNYI TELRRQHARA SHLGLA
M9192: Haptoglobin alpha-1 chain
VDSGNDVTDI ADDGCPKPPE IAHGYVEHSV RYQCKNYYKL
RTEGDGVYTL NNEKQWINKA VGDKLPECEA VCGKPKNPAN PVQ
M10069: Apolipoprotein A1 (C-terminal fragment)
AHVDALR THLAPYSDEL RQRLAARLEA LKENGGARLA
EYHAKATEHL STLSEKAKPA LEDLRQGLLP VLESFKVSFL
SALEEYTKKL NTQ
M28284: Apolipoprotein A1
DEPPQSPWDR VKDLATVYVD VLKDSGRDYV SQFEGSALGK
QLNLKLLDNW DSVTSTFSKL REQLGPVTQE FWDNLEKETE
GLRQEMSKDL EEVKAKVQPY LDDFQKKWQE EMELYRQKVE
PLRAELQEGA RQKLHELQEK LSPLGEEMRD RARAHVDALR
THLAPYSDEL RQRLAARLEA LKENGGARLA EYHAKATEHL
STLSEKAKPA LEDLRQGLLP VLESFKVSFL SALEEYTKKL NTQ
M81763: Transferrin
VPDKTVRWCAVSEHEATKCQSFRDHMKSVIPSDGPSVACVKKASYLDCIR
AIAANEADAVTLDAGLVYDAYLAPNNLKPVVAEFYGSKEDPETFYYAVAV
VKKDSGFQMNQLRGKKSCHTGLGRSAGWNIPIGLLYCDLPEPRKPLEKAV
ANFFSGSCAPCADGTDFPQLCQLCPGCGCSTLDEYFGYSGAFKCLKDGAG
DVAFVKHSTIFENLANKADRDQYELLCLDNTRKPVQDYKDCHLAEVPSHT
VVARSMGGKEDLIWELLNQAQEHFGKDKSKEFQLFSSPHGKNLLFKDSAH
GFLKVPPRMNAKMYLGYEYVTAIRNLREGTCPEAPTNECKPVKWCALSHH
ERLKCNEWSVSDVGKIECVSAETTEDCIAKIMNGEADAMSLDGGFVYAIG
KCGLVPVLAENYNKSDDCEQTPADGYFAVAVVKKSASDLTWDNLKGKKSC
HTAVGRTAGWNIPMGLLYNKINHCRFDEFFSEGCAPGSKKDSSLCKLCMG
SGLNLCEPNNKEGYYGYTGAFRCLVEKGDVAFVKHQTVTQNPGGKNPDWP
AKDLNEKYNELCLDGTRKPVQEYANCHLARAPNHAVVTRKDKEACVHKIL
RQQQHLFGSNVTDCSGNFCLFRSETKDLLFRDDTVCLAKLHDRNTYEKYL
GQEYVKAVGNLRKCSTSSLLEACTFRRP
(As recited in Ross et al., Proc. Natl. Acad. Sci.
USA, 79: 2504-2508 (1982))

Thus, M9192 and M81763, which were upregulated in serum patients with subsequent metastatic relapse were identified as Haptoglobin alpha 1 chain and Transferrin, respectively, while M8936, which was positively correlated to metastasis-free survival, was shown to be C3a complement fraction. Additionally, M28284 and M6647 were identified to be Apolipoprotein A1 and Apoliprotein C1, respectively, which low expression was associated to metastatic relapse (data not shown).

What is claimed is:

1. A method for determining breast cancer status in a subject comprising:
(a) measuring at least one biomarker in a biological sample from the subject, wherein the at least one biomarker is selected from the group consisting of the biomarkers of Table 1; and
(b) correlating the measurement with breast cancer status.

2. The method of claim 1, wherein breast cancer status is relapse of breast cancer versus breast cancer free survival.

3. The method of any of claims 1-2, wherein the at least one biomarker is measured by capturing the biomarker with a capture reagent on an adsorbent surface of a SELDI probe and detecting the captured biomarker by laser desorption-ionization mass spectrometry.

4. The method of claim 3, wherein the capture reagent comprises an antibody.

5. The method of claim 3, wherein the capture reagent comprises an IMAC or CM10 sorbent.

6. The method of any of claims 1-2, wherein the at least one biomarker is measured by immunoassay.

7. The method of any of claims 1-2, wherein the sample is serum.

8. The method of any of claims 1-2, wherein the correlating is performed by a software classification algorithm.

9. The method of any of claims 1-2, further comprising: (c) managing subject treatment based on the status.

10. The method of claim 9, further comprising: (d) measuring the at least one biomarker after subject management and correlating the measurement with disease progression.

11. A method for determining the course of breast cancer comprising:
(a) measuring, at a first time, at least one biomarker in a biological sample from the subject, wherein the at least one biomarker is selected from the group consisting of the biomarkers of Table 1;
(b) measuring, at a second time, the at least one biomarker in a biological sample from the subject; and
(c) comparing the first measurement and the second measurement;
wherein the comparative measurements determine the course of breast cancer.

12. A method comprising measuring at least one biomarker in a sample from a subject, wherein the at least one biomarker is selected from the group consisting of biomarkers of Table 1.

13. A composition comprising at least one purified biomolecule selected from the biomarkers of Table 1.

14. A composition comprising a biospecific capture reagent that specifically binds a biomolecule selected from the biomarkers of Table 1.

15. The composition of claim 14, wherein the biospecific capture reagent is an antibody.

16. The composition of claim 14, wherein the biospecific capture reagent is bound to a solid support.

17. A composition comprising a biospecific capture reagent bound to a biomarker of Table 1.

18. A kit comprising:
(a) a solid support comprising at least one capture reagent attached thereto, wherein the capture reagent binds at least one biomarker selected from the group consisting of the biomarkers of Table 1; and
(b) instructions for using the solid support to detect a biomarker of Table 1.

19. The kit of claim 18 wherein the solid support comprising a capture reagent is a SELDI probe.

20. The kit of claim 18 wherein the capture reagent is an antibody.

21. The kit of claim 18, additionally comprising: (c) a container containing at least one of the biomarkers of Table 1.

22. The kit of claim 18, additionally comprising: (c) a strong cation exchange chromatography sorbent.

23. A kit comprising:
(a) a solid support comprising at least one capture reagent attached thereto, wherein the capture reagent binds at least one biomarker selected from the group consisting of the biomarkers of Table 1; and
(b) a container containing at least one of the biomarkers.

24. The kit of claim 23 wherein the solid support comprising a capture reagent is a SELDI probe.

25. The kit of claim 23 additionally comprising: (c) a strong cation exchange chromatography sorbent.

26. The kit of claim 23 wherein the capture reagent is an antibody.

27. A software product comprising:
a. code that accesses data attributed to a sample, the data comprising measurement of at least one biomarker in the sample, the biomarker selected from the group consisting of the biomarkers of Table 1; and
b. code that executes a classification algorithm that classifies the breast cancer status of the sample as a function of the measurement.

28. The software product of claim 27, wherein the data comprises measurement of all of the biomarkers of Table 1.

29. A method comprising detecting at least one biomarker of Table 1 by mass spectrometry or immunoassay.

30. A method comprising communicating to a subject a diagnosis relating to breast cancer status determined from the correlation of at least one biomarker in a sample from the subject, wherein said at least one biomarker is selected from the group consisting of the biomarkers of Table 1.

31. The method of claim 30 wherein the diagnosis is communicated to the subject via a computer-generated medium.

32. A method for identifying a compound that interacts with a biomarker of Table 1, wherein said method comprises:
a) contacting a biomarker of Table 1 with a test compound; and
b) determining whether the test compound interacts with the biomarker.

33. A method for modulating the concentration of a biomarker of Table 1 in a cell, wherein said method comprises contacting said cell with a compound that modulates the expression of the biomarker.

34. A method of treating breast cancer in a subject, comprising administering to the subject a therapeutically effective amount of a compound that inhibits expression of an up-regulated biomarker of Table 1.

35. A method of treating breast cancer in a subject, comprising administering to the subject a therapeutically effective amount of a compound that increases expression of a down-regulated biomarker of Table 1.


SEQUENCE LISTING (7 sequences)

SEQ ID NO: 1 - protein, 55 aa, Unknown
  Description of Unknown Apolipoprotein C-I biomarker sequence
DVSSALDKLKEFGNTLEDKARELISRIKQSELSAKMREWFSETFQKVKEKLKIDS

SEQ ID NO: 2 - protein, 57 aa, Unknown
  Description of Unknown Apolipoprotein C-I biomarker sequence
TPDVSSALDKLKEFGNTLEDKARELISRIKQSELSAKMREWFSETFQKVKEKLKIDS

SEQ ID NO: 3 - protein, 76 aa, Unknown
  Description of Unknown Complement C3a biomarker sequence
SVQLTEKRMDKVGKYPKELRKCCEDGMRENPMRFSCQRRTRFISLGEACKKVFLDCCNYITELRRQHARASHLGLA

SEQ ID NO: 4 - protein, 83 aa, Unknown
  Description of Unknown Haptoglobin alpha-1 chain biomarker sequence
VDSGNDVTDIADDGCPKPPEIAHGYVEHSVRYQCKNYYKLRTEGDGVYTLNNEKQWINKAVGDKLPECEAVCGKPKNPANPVQ

SEQ ID NO: 5 - protein, 90 aa, Unknown
  Description of Unknown Apolipoprotein A1 biomarker sequence
AHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 6 - protein, 243 aa, Unknown
  Description of Unknown Apolipoprotein A1 biomarker sequence
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 678 aa, Unknown
  Description of Unknown Transferrin biomarker sequence
VPDKTVRWCAVSEHEATKCQSFRDHMKSVIPSDGPSVACVKKASYLDCIRAIAANEADAVTLDAGLVYDAYLAPNNLKPVVAEFYGSKEDPETFYYAVAVVKKDSGFQMNQLRGKKSCHTGLGRSAGWNIPIGLLYCDLPEPRKPLEKAVANFFSGSCAPCADGTDFPQLCQLCPGCGCSTLDEYFGYSGAFKCLKDGAGDVAFVKHSTIFENLANKADRDQYELLCLDNTRKPVQDYKDCHLAEVPSHTVVARSMGGKEDLIWELLNQAQEHFGKDKSKEFQLFSSPHGKNLLFKDSAHGFLKVPPRMNAKMYLGYEYVTAIRNLREGTCPEAPTNECKPVKWCALSHHERLKCNEWSVSDVGKIECVSAETTEDCIAKIMNGEADAMSLDGGFVYIAGKCGLVPVLAENYNKSDDCEQTPADGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVTQNPGGKNPDWPAKDLNEKYNELCLDGTRKPVQEYANCHLARAPNHAVVTRKDKEACVHKILRQQQHLFGSNVTDCSGNFCLFRSETKDLLFRDDTVCLAKLHDRNTYEKYLGQEYVKAVGNLRKCSTSSLLEACTFRRP